(19) United States
(12) Patent Application Publication
(10) Pub. No.: US 2015/0186829 A1
(43) Pub. Date: Jul. 2, 2015

(54) COORDINATING A MULTI-STEP TASK AMONG ONE OR MORE INDIVIDUALS

(71) Applicants: UMAIR KHAN, MILTON (CA); HUGO MACHADO, OAKVILLE (CA)

(72) Inventors: UMAIR KHAN, MILTON (CA); HUGO MACHADO, OAKVILLE (CA)

(21) Appl. No.: 14/315,808

(22) Filed: Jun. 26, 2014

Related U.S. Application Data
(60) Provisional application No. 61/921,941, filed on Dec. 30, 2013.

(51) Int. Cl. G06Q 10/06; G06Q 10/10

(52) U.S. Cl. CPC G06Q 10/06316; G06Q 10/063114; G06Q 10/103

(57) ABSTRACT

An event notification system (ENS) is in communication with a hospital information system (H.I.S.) over a local network. The hospital information system supports a patient scheduling system, one or more hospital staff call systems and a patient information system. The ENS uses information stored in all of the systems supported by the H.I.S. to coordinate the hospital staffs activities associated with the transportation of a patient from one location to another.

### CROSS REFERENCE TO RELATED APPLICATIONS

This application claims the benefit under 35 U.S.C. §119(e) of U.S. Provisional Patent Application Ser. No. 61/921,941 entitled “Patient Transportation System”, filed Dec. 30, 2013, the entire contents of which are incorporated herein by reference.

### BACKGROUND

1. Field of the Invention

The present disclosure relates to event notification systems that generate and send an event notification message to an individual requesting that that a task be performed and tracking the current state of the task.

2. Background

In organizations that employ multiple individuals that need to coordinate their activities to effectively perform a task, it is helpful to use a notification system that operates to generate and send notification messages both alerting the individuals to a new or outstanding task that needs to be performed and tracking the state of the performance of the task. Such an automated system can be used in a clinical setting, a hospitality setting, an airport setting, a property management of emergency management setting to name just a few. Such an automated system can receive a request from an individual or a scheduling system, for instance, that includes information about a particular task, activity or condition that needs to be performed, and the system can then make a determination regarding which individual(s) should be notified that a task is outstanding, and send a message to the individual(s) that includes instructions relative to the performance of the task.

Typically, such a notification system can operate to receive some sort of acknowledgement from the notified individual(s) indicating that they are, or are not, able to perform the requested task. If the notified individual responds by indicating that they are able to perform the task, the notification system generally assumes that the task will be performed and requires no further confirmation. Other notification systems may run a process that tracks the status of an outstanding task until it is performed, and request that the individual or individuals responsible for performing the task send periodic updates.

### BRIEF DESCRIPTION OF THE DRAWINGS

The present invention can be best understood by reading the specification with reference to the following figures, in which:

FIG. 1 is a diagram illustrating a hospital information system.

FIG. 2 is a diagram illustrating functional elements comprising an event notification system 110.

FIG. 3 is a diagram showing functional elements comprising a schedule processing & notification function 210.

FIG. 4A is an illustration of a nurse task request message.

FIG. 4B is an illustration of options comprising the nurse task request message.

FIG. 4C is an illustration of a task status message.

FIG. 5A is an illustration of a porter task request message.

FIG. 5B is an illustration of options comprising the porter task request message.

FIG. 5C is an illustration of a task status message.

FIG. 5D is an illustration of the options shown in FIG. 5B.

FIG. 5E is an illustration of a task status message.

FIG. 5F is an illustration of a porter task request message.

FIG. 5G illustrates options available to a porter in the task request of FIG. 5F.

### DETAILED DESCRIPTION

While notification systems associated with a scheduling application can operate to locate and notify an individual that can be responsible for completing a task, these systems typically only ensure that an individual is notified of an outstanding task to be performed, and then detect that the task is performed. Current notification systems are not able to track the current status of a complex task requiring that a plurality of steps be successfully performed in order to complete the task. Such notification systems are further limited in as much as they are not able to coordinate the performance of each step of a multi-step task between two or more individuals responsible for the performance of different steps in the multi-step task. These and other limitations of current notification systems are overcome in an embodiment of an event notification system (ENS) that operates to send a multi-step task request message to an individual, and then operating to track the performance of each step in the multi-step task to the successful completion of the task. In another embodiment, an ENS operates in conjunction with a scheduling application to notify two or more individuals of an outstanding multi-step task, and then to coordinate the completion of each step of the task between the two or more individuals.

While the following description discloses embodiments of the invention in the context of a clinical environment, it should be understood that other embodiments of the invention can be implemented in security environments, hospitality environments, airport environments or property management environments to name on a few. Generally, the invention can be implemented in any environment in which one or more individuals are notified by an event notification system (ENS) that a scheduled, multi-step task is to be completed, and then interacting with the ENS after performing each of the multiple steps associated with the task.

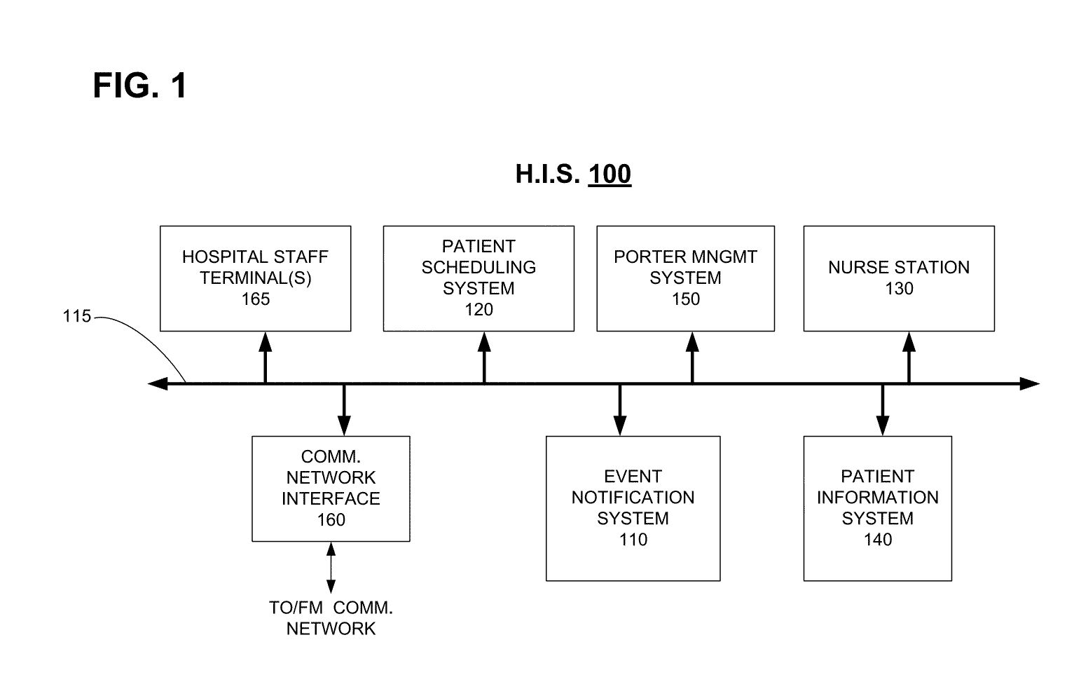

Several functional elements comprising a clinical or hospital information system (HIS) 100 are shown in FIG. 1. An event notification system (ENS) 110 is in communication over a local network 115 with a patient scheduling system/application 120, a nurse station 130, a patient information system 140, a porter management system 150, one or more staff terminals/computers 165, and with one or more interfaces 160 to a wireless and/or a wired communication network. The design and operation the patient scheduling system 120 is well known to those who are familiar with the design of hospital scheduling or other types of scheduling systems. Generally, a network administrator can grant an individual (task requestor) permission to access the scheduling system or any of the other systems connected to the network 115. For example, imaging department staff, (task requestor) can be granted permission to access the scheduling system 120, via any one of the terminals 165, to enter information into a task schedule regarding the scheduling of a patient's test or medical procedure (task) at a particular time and at a particular place, and they can enter any special considerations relative to the service being provided to the patient at the scheduled time (such as special patient preparation procedures administered by a nurse or nurses aid, and any special transportation considerations performed by a porter or some other staff member). Using a terminal 165, a staff member can access patient information stored in the patient information system 140, they can access the name or names of the nurses and/or doctors currently assigned to the patients care, and they can access other patient information such as the patients current location (building, wing, floor, ward, etc). Alternatively, a patient scheduling application can be implemented to run in the ENS 110, and selected hospital staff (requestors) are given permission to log in to the ENS in order to schedule patient tests and/or procedures and to access patient information as described above.

The nurse station 130 in FIG. 1 can be comprised of a plurality of different devices running applications that assist the nursing staff with the management of patient care, and to generally communicate with other staff members. The station 130 can have, among other things, a nurse call function, a nurse assignment function, a nurse location function, a communication terminal function, and a nurse status function. The nurse call function generally operates as a communication device between a patient and a nurse. Various events associated with a patient can cause a message to be sent to the call station indicating that the patient needs to be tended to in some manner. The nurse assignment function includes a list of nurses who are currently on-duty and the names of patients that are assigned to them. The nurse location function tracks the current location of each on-duty (and/or off-duty) nurse, and the nurse status function tracks whether or not a nurse is currently performing a task (busy or free) and the nature or criticality level of the task that is being performed.

Multiple nurse stations 130 can be strategically located throughout a clinical setting and operate, via the communication terminal functionality, as an interface to the various elements comprising the hospital information system 100. The station 130 can receive messages from any of the other hospital systems indicating that a patient is being admitted to the hospital, the station 130 can, as described earlier, receive messages directly from patients (patient needs assistance) or receive telemetry from a patient's bed including information corresponding to a patient's clinical condition, the system 130 can be used by the nursing staff to access the scheduling system 120, and the system 130 can be employed to call for assistance from other hospital staff members (using either the LAN or WAN interfaces.

The patient information system 140 stores information corresponding to each patient that is currently admitted to the hospital or patients who have been admitted in the past. Among other things, the system 140 can include the building, wing, floor and number of a room to which a patient is currently assigned. The system 140 can include clinical information associated with current and past testing or diagnosis, the names of medical staff who have treated a patient, and any other relevant information.

The porter management system 150 generally operates to, among other things, maintain a list of porters that are on duty, currently available to move patients around a hospital and the current physical location of each porter in the hospital. The system can make this porter information available to the other hospital systems as needed. Porters can log in to the system 150 when they come on duty and they can log out of the system when they go off duty. The system 150 can include functionality to track the location of a porter in a hospital during the time that they are on duty. The ENS 110 generally operates to receive or retrieve information from other hospital systems, to process the information to determine what action should be taken by hospital staff, send an alert/notification message to the appropriate hospital staff (doctor, nurse, technician, porter, others) that can include a request to attend to a patient in some manner (preparation for transport or transportation for instance), a request for assistance or some other relevant action, and the ENS 110 can operate to receive a response to the notification message from the hospital to attend to the patient. The notification message can be sent to hospital staff via either the local network 115 or wide area communication network. It should be understood, that although the ENS 110 is shown as a standalone system in FIG. 1, that it can comprise any of the other systems that are included in the H.I.S. 100. In this regard, the ENS can comprise, among other things, the porter management system 150 and the patient scheduling system 120. The event notification system 110 described above with reference to FIG. 1 is described in both U.S. patent application Ser. No. 13/347,380 entitled “EVENT NOTIFICATION SYSTEM FOR ASSOCIATING AN OUTGOING ELECTRONIC MESSAGE WITH AN INCOMING RESPONSE”, filed Jan. 10, 2012, and U.S. patent application Ser. No. 13/757,117 entitled “EVENT NOTIFICATION SYSTEM FOR ALERTING THE CLOSEST APPROPRIATE PERSON” the entire contents of both applications of which are incorporated herein by reference.

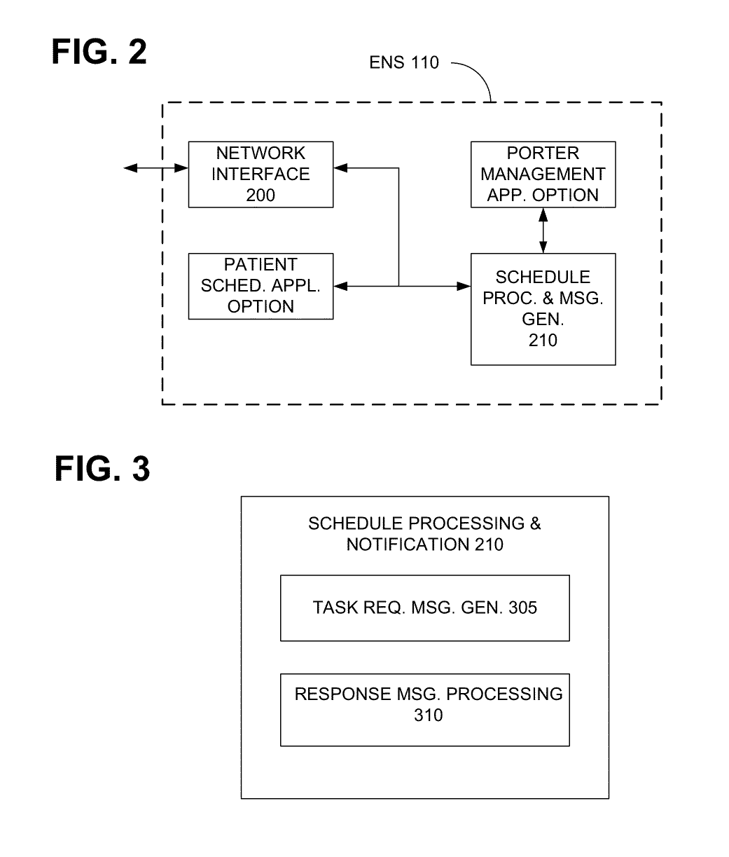

FIG. 2 illustrates functionality comprising an event notification system such as the ENS 110 described earlier with reference to FIG. 1. In general operation, the ENS 110 can receive or retrieve event messages or patient scheduling information over an interface 200 from an event generation device (scheduling system for instance), and a schedule processing and message generation module or simply message generation module 210 can use the received event or scheduling information to determine whether to generate and to which individual or individuals to send a task request message, and then determine what information to place into the message.

As described above with reference to FIG. 2, the message generation module 210 operates to send a task request message to one or more individuals, and it also operates to receive a response message from one or more of these individuals. Upon receiving a response from an individual, the ENS 110 can parse information comprising the response to determine which task request message the response relates to, it can determine from which individual the response is received, and it can identify particular information included in the response.

As described later with reference to FIG. 3, and according to one embodiment, the ENS 110 operates to receive scheduling information from the scheduling system 120, and operates on this information to generate one or more messages requesting that a multi-step task be performed. The ENS includes functionality that operates to determine from the scheduling information whether or not one or more individuals should be notified, and it can determine which individuals or types of individuals (nurse, porter, other) should be sent a request to perform one or more steps of a multi-step task (i.e., that a particular patient is scheduled for a test or for a procedure in a particular department at a particular time). The ENS 110 can operate to determine which of a plurality of options can be included in a task request message. These options are specific to each particular type of task and are displayed on a device (mobile or stationary device screen) for selection by the individual to which the task request message is sent. Options included in a task request message can include, but are not limited to, the following: option to request one porter only, option to request one porter with a wheel chair or a stretcher, option to request two porters, option indicating that a patient is not ready.

The ENS can also determine whether the patient should to be prepared in some special/specified manner prior to being transported, or whether the patient has special transportation requirements, and to determine who and when hospital staff should be notified of the patients transportation needs. Some or all of the determinations arrived at by the ENS can be included in instructions comprising the one or more task request messages which can be sent to a stationary device connected to the network 115 or to a mobile device able to connect to the network to communicate with the ENS 110. The scheduling system or application from which the ENS receives patient scheduling information can be associated with a particular clinical department (X-ray imaging for instance), and departmental staff (requestor) can create a patient appointment entry in the scheduling system 120 (daily, weekly or longer) that includes patients names, medical record number (MRN), times that they are due for testing/imaging (for instance), the location to which they should be transported, whether the patient should be prepared prior to transportation, and any other special/optional requirements (patient prep or special transportation needs).

FIG. 3 is a block diagram illustrating functional elements comprising the message generation module 210 comprising the ENS 110 described earlier with reference to FIGS. 1 and 2. The module 210 comprises logical instructions that when operated on by a computational device associated with the ENS 110 control the module 210 to access and process information stored on any of the devices or systems comprising the H.I.S. 100 to generate and send a task request message to one or more individuals or departments, and the instructions also control the module 210 to receive response messages from individuals and to process information comprising the response messages to determine what further task request or requests are to be generated. These logical instructions are implemented, according to one embodiment, in a task request message generation function 305 and in a response message processing function 310.

The task request message generation function 305 comprising the module 210 operates to receive or retrieve information from any of the systems or devices connected to the local network 115 and to use this information to generate task request messages. Among other things, it can access or receive information stored in the patient scheduling system 120, it can access information stored in the porter management system 150, it can access information stored in the patient information system 140, and it can access information stored in the nurse station 130. The message generation function 305 operates on the information received from the H.I.S. 100 to identify scheduling information (time, patient name, patient location) and patient status information, to identify individuals to which task requests are sent, and to identify any other information (requesting department, priority, medical record number, additional information such as special transportation or patient prep considerations) used to generate a task request message that is sent to one or more individual staff members. In one embodiment, the message is a request for patient transportation. The task request message generation function 305 generally operates to either generate a request message for immediate transmission to the appropriate staff member, or to generate a message that is marked for transmission at some later time (i.e., the time at which a message is transmitted can be programmed to be X minutes prior to the time a patient is scheduled for transport). The function 305 determines, based upon the current status of a scheduled activity, what information is included in a task request message and to which individual the request is sent. For instance, if the message is an initial message sent to a nurse requesting that they prepare a patient for transportation in thirty minutes, then the message can include the patient and test/procedure scheduling information as well as any special patient transportation or preparation needs (patient needs to be transported in a wheel chair, patient needs to be transported while connected to life support equipment, etc.). Subsequent to the nurse responding to the initial request message with an indication that they have completed their task or scheduled activity, and subject to any options selected by the nurse that are included in their response, the ENS 110 (module 305 specifically) can generate and send a task request message to an appropriate porter that includes all of the information the porter needs to transport the patient. Among other things, the information comprising the message (or transportation request message) sent to the porter or porters can depend upon the options that are selected by the nurse in their response to the notification message they receive (first notification message).

The response message processing function 310 shown in FIG. 3 can operate to receive responses to a task request message from individuals, and it can parse the information received in the response messages to determine from which individual the message is sent and to determine what information is included in the message. For instance, the function 310 can determine that a response message is from a nurse currently responsible for a particular step in a task that is as yet not completed. It can determine that the response message includes information indicating that the nurse selected a particular option, and it can then pass this information (individuals ID, patient information, status, options selected, etc.) to the message generation function 305 which can operate to generate a task request message that can be sent to, for instance, a porter.

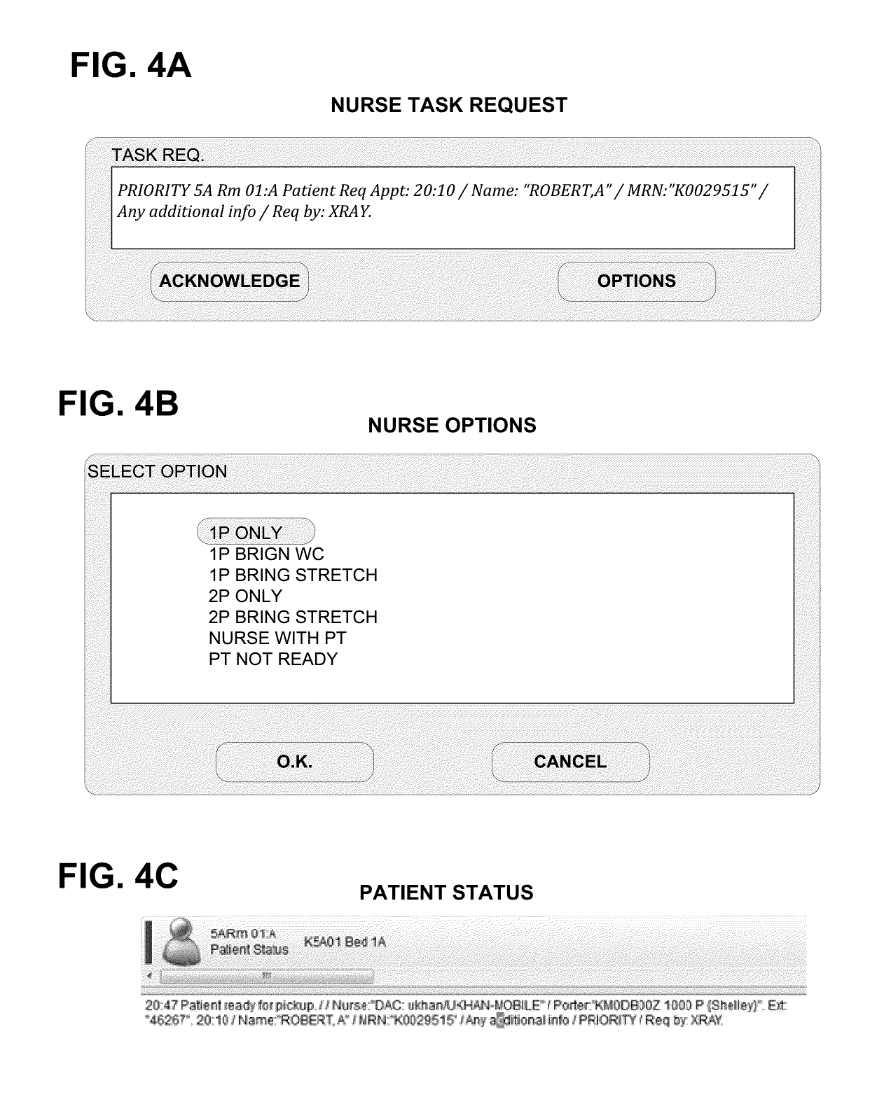

As described above, the ENS 110 can access information comprising the scheduling system 120, or any of the other systems connected to the local network 115 and comprising the H.I.S. 100, to determine which individual or individuals should receive a multi-step task request message or simply task request message. Such a task request message can be generated and sent to nurses, porters or any other individual working in a clinical setting, FIG. 4A illustrates a task request message sent to a nurse. The task request message can include, but is not limited to, the request priority, the patient's name, the patient's location, the department to which the patient is to be transported, the scheduled test/procedure time, whether the patient needs special preparation, The task request message can also have standard or custom response options (see FIG. 4B below) that a nurse can select, such as whether they are unable find the transport means at the suggested location or whether the porter has arrived. The message can remain active until such time as the nurse has completed their task or the task is transferred to another nurse. In operation, the ENS 110 determines the identity and availability of a nurse assigned to a patient using information received from the nurse station 130 or the patient information system 140, and is able to determine which available porter or porters are the appropriate ones to notify using information received from the porter management system 150. In the event that a nurse is notified to perform some task in a task request message, they can be prompted to respond to the task request message by selecting, in this case, the acknowledge button displayed in the request message. Responding in this manner indicates that they are able to perform the task of, for instance, preparing the patient for transport. This task request message can include two or more options that are specific to the task, such as the options shown in FIG. 4B. Subsequent to completing any required patient preparation, the nurse can select one of the two or more options that are sent to them in the task request message, and which can be displayed on either a mobile communications device or a stationary computer terminal by selecting the “options” button displayed in the task request message as shown in FIG. 4A. As a result of the ENS involving the nurse in the process of performing the task, the nurse is able to control the selection of the most appropriate patient transportation means (e.g. with wheelchair, stretcher, 2 porters, etc) by selecting one of the options made available to them in the message. Once the options are displayed and one is selected (in this case the option “1P ONLY”), the nurse can select “O.K.” and a message is sent from their device to the ENS 110 indicating that the nurse has completed the step of preparing the patient for transport. After the nurse has indicated that the patient is ready for transport, the ENS can generate and transmit/broadcast a patient or task status information message (illustrated in FIG. 4C) to an appropriate nurse station, to the department to which the patient is scheduled for testing or any other device comprising the H.I.S. 100.

Continuing to refer to FIG. 4A, if the nurse selects the acknowledge button, but then does not subsequently select an options button, the ENS 110 determines that the step of preparing the patient for transport has not been completed by the first nurse, and the ENS can escalate the task by either sending another, similar task request message to the same (first) nurse, or by sending a task request message to a different, second nurse and cancel the task request message originally sent to the first nurse. Further, the ENS 110 can escalate the task request message by generating another request to the second nurse after some predetermined period of time (threshold time duration) which can be configured by a system administrator, a nurse, or any other person with permission to configure the ENS. In the event that the nurse is responsible to transporting a patient, and they notify the ENS that they have completed this step of the requested task, then the ENS can determine that all of the steps of the task are complete. On the other hand, if a porter is needed for patient transportation, then another task request message can be generated.

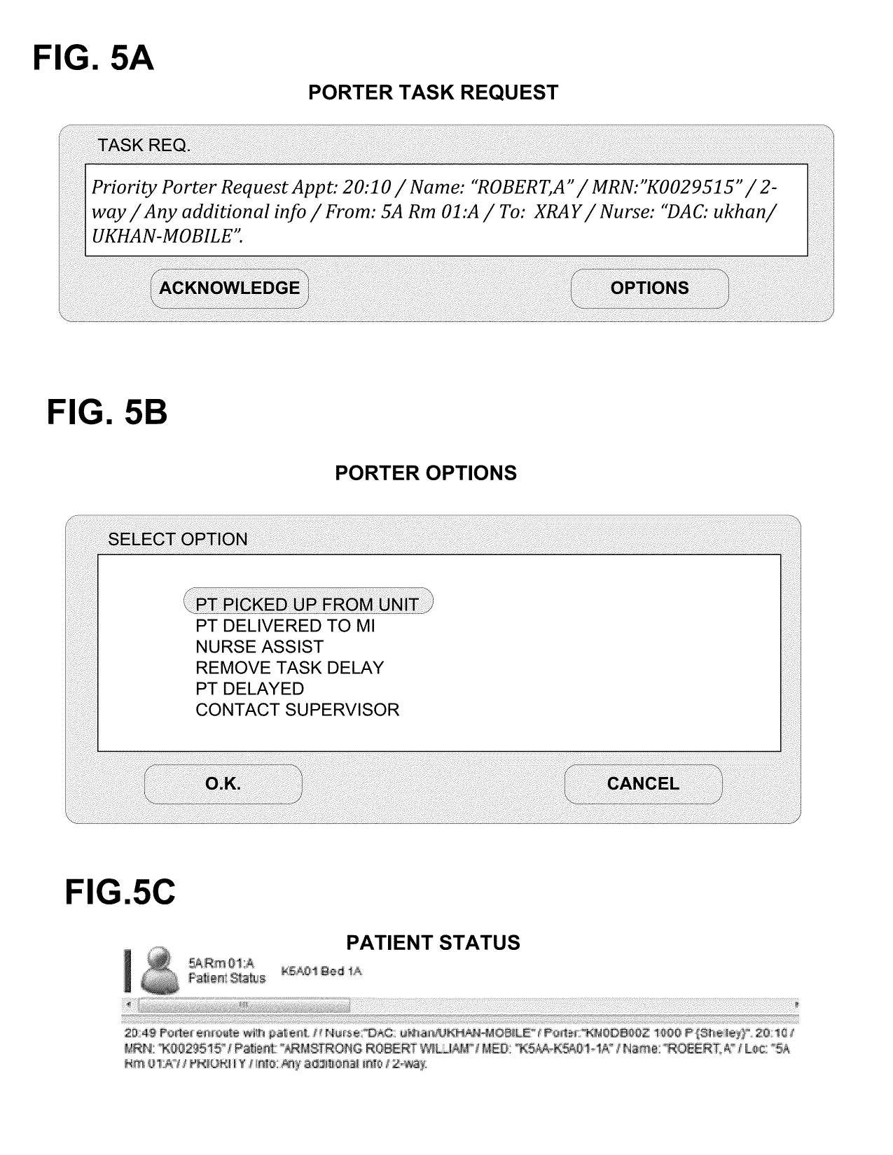

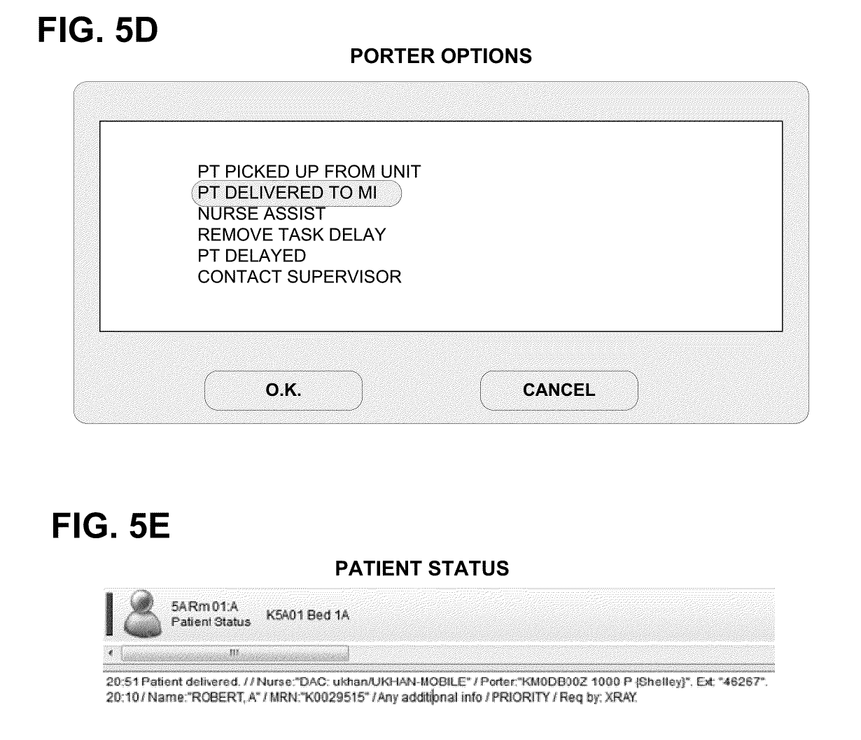

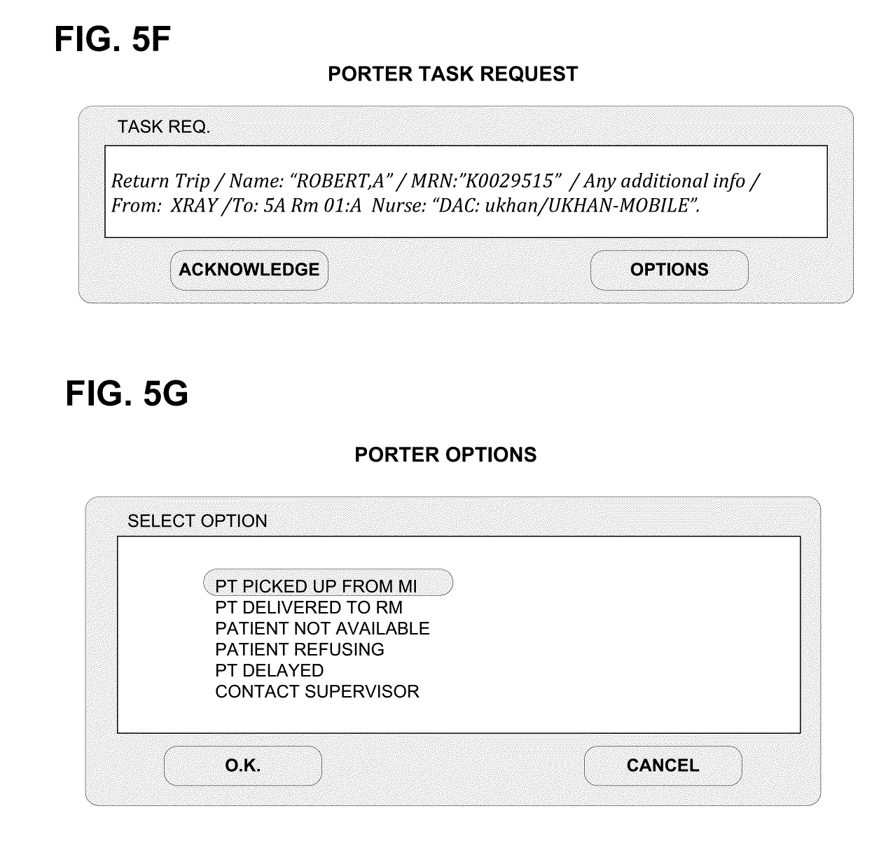

Subsequent to the nurse completing their portion of the task, the ENS 110 can send a task request message to one or more porters requesting them to complete the task of transporting the patient to their scheduled test or procedure. This request, illustrated with reference to FIG. 5A, can be sent to the porter who may have assisted the nurse to prepare the patient, or it can be sent to another available porter. Each time a hospital staff member receives a task request message, they are prompted to respond, and the ENS reacts to the response in some appropriate manner (i.e., it escalates the notification, notes which staff member responded and how they responded, updates a patient status). Also, each time a task or a step in the task is completed, the individual who is responsible for completing the task or the task step can select an option that is included in the task request message which causes a message to be sent to the ENS 110 with information indicating that a task is completed, not completed or that another step of the task is waiting to be assigned. Once all of the steps associated with a task are completed, the ENS broadcasts a message to some or all of the devices comprising the H.I.S. 100 indicating that the task has been completed. On the other hand, if all of the steps in a task have not been completed, the ENS can generate another message, such as the task request message illustrated in FIG. 5A and described below.

The message shown in FIG. 5A can have all of the information that a porter needs in order to transport a patient. The ENS 110 can access information stored in any of the systems comprising the H.I.S. 100 in order to determine which one or more porters are the appropriate ones to send the message to. The task request message can include, but is not limited to, the request priority, the patient's name, the patient's location, the department to which the patient is to be transported, the scheduled test/procedure time, the time it takes to transport the patient from their current location to the department or procedure location, the means by which the patient is to be transported, whether the porter is to transport the patient and wait (and for how long they should wait), or transport the patient, leave and return (and at what time they should return) or not return, and any special considerations for the patients transportation (need for a particular type of transport means, such as a wheel chair, gurney, and the current location of these transport means within the clinical facility). The task request message can also have standard or custom response options that a porter can select, such as the time they will start transporting the patient, whether there is any delay during transit, and whether they are unable find the transport means at the suggested location. The message can remain active until such time as the porter has completed their task or the task is transferred to another porter. After receiving the task request message, the porter is prompted to either acknowledge its receipt and/or the porter can select one option from among a listing of options included in the message. After receiving a response to the task request message from the porter, the ENS 110 can broadcast a patient or task status message (FIG. 5C) to the nurse and to the department to which the patient is being transported that includes information relating to the transportation of the patient, such as the patient is in transit and the estimated patient arrival time. The transit time can be an estimated value entered into the ENS by an a system administrator or it can be a value that is arrived at empirically (learned) by the system or staff/requestor. The department to which the patient is being transported can respond to the patient status message with an indication that, among other things, they are servicing patients according to their scheduled times or not. If the department to which the patient is being transported is experiencing delays, departmental staff can respond to any of the task request messages by indicating that the patient transport should be delayed for a specified period of time. As described above, a task request message can include a message priority. In this regard, the generation module 305 described with reference to FIG. 3 can operate to access information stored on devices and systems comprising the H.I.S. 100 to determine a message priority. The message priority can be determined according to the department requesting that a task be performed, the time/date on which the task is requested, the condition of the patient associated with the task, or any other relevant information.

When the porter has delivered the patient they are transporting to the medical imaging department, they are prompted to select the “PT DELIVERED TO MI” option button displayed on their mobile device screen and illustrated in FIG. 5D. Selecting this option sends a task status message (illustrated in FIG. 5E) to the ENS 110 that the patient has been successfully transported to the MI department and that this step of the task is complete. The ENS can then notify the responsible nurses and/or MI department staff (or any other interested party) that the patient has been delivered for imaging or testing (patient status message). At the point in time that the imaging department is finished with the patient, a member of the department can send an indication to the ENS that the patient is ready for transport back to their room. If the original request for transportation specifies that the porter is responsible to transport the patient back to their room (2-way portering), then the ENS can generate and send another task message, illustrated in FIG. 5F, to the original porter requesting that they transport that patient back to their room. In this regard, the ENS 110 stores a history of messages that it transmitted and responses it receives from the porter or others and is able to use information comprising these messages to determine to whom the next task request message is sent and what information is included in the message. Alternatively, if the porter that originally transported the patient is not requested to wait (1-way portering), then the ENS sends a request message to the porting system in order to identify another porter who is available to transport the patient. Regardless, after receiving the task request message illustrated in FIG. 5F, a porter can acknowledge receipt and then elect to display the options, which are shown in FIG. 5G. Provided the porter selects the option “PT PICKED UP FROM MI”, a message is sent to the ENS indicating that the patient is in transit back to their room. Subsequent to the porter delivering the patient to their room, they can select the “PT DELIVERED TO RM” option. Selecting this option sends a task status message (note shown) to the ENS 110 that the patient has been successfully transported back to their room and that this step of the task is complete. The ENS can then notify the responsible nurses and/or MI department staff (or any other interested party) that the patient has been delivered to their room and the ENS can then notify all interested parties of the status of the task, namely, that the multi-step task is complete, at which point the task can be cancelled from the scheduling system and marked as completed at the ENS.

Each notification message generated by the ENS 110 and transmitted to a staff member can remain active until one or more or the requested task step activities are performed or completed. Among other things, a task message to a nurse can request that they prepare a patient for transportation at a particular time. Referring again to FIG. 4A, the task request message can include the priority level of the request, the name and room number of the patient, the date and time at which the patient is scheduled for a procedure or test, the department/location to which the patient is to be transported and it can have any special instructions for the preparation of the patient (i.e., the patient should remain connected to oxygen or IV fluids or monitors). As described earlier, the task request message can prompt the nurse to respond by either simply acknowledging receipt of the message or by selecting an option to be included in their response or both. Each task request message generated by the ENS that is associated with a different type of task can have a different set of one or more response options that the ENS is able to process to determine what operation (task step) to perform next, such as generating and sending a message to a porter notifying them that the patient is ready for transport. Alternatively, the nurse can enter a non-standard response (either text or speech) into the system that the system processes using either a text recognition application or a speech recognition application, and the ENS can then use the response to determine the next appropriate step. If they respond by selecting an option indicating that the patient is not yet ready for transport, the ENS 110 can operate to wait a period of time before sending another, second message (status request message) requesting that the nurse provide some indication regarding the status of the patient preparation process, or the first message can include an option or options that a nurse can select indicating how long it will take them to prepare the patient (5 minute, 10 minutes, etc). This second message can be transmitted at a time that corresponds to the preparation time option selected by the nurse in responding to the first message (i.e., if the nurse responds at 12:00 and selects an option indicating that it will take them 10 minutes to prepare the patient, then the second message can be sent to the nurse at 12:10). Additionally, the first or second message can include an option that a porter be notified that the patient needs to be transported, or the message can include an option indicating that the nurse is transporting the patient. This allows the nurse to select the appropriate patient transport means (e.g. with wheelchair, stretcher, 2 porters, etc). The dispatch method can vary depending on the workflow requirements, centralized dispatching, automated dispatching, tiered dispatching, etc. As described above, each task message can include an indication of the priority of the message.

Several embodiments of an ENS system have been described that operate to generate and transmit multi-step task request messages to one or more individuals. It operates to track the status of each multi-step task until the task is completed and it operates to broadcast the status of the task's completion to a hospital information system. Each individual receiving a task request message can respond by selecting an option from a set of options that is particular to that task, and the ENS 110 determines base upon information received from individual responses whether to generate additional task request messages. The forgoing description, for purposes of explanation, used specific nomenclature to provide a thorough understanding of the invention. However, it will be apparent to one skilled in the art that specific details are not required in order to practice the invention. Thus, the forgoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

We claim:

1. A method for coordinating the performance of a multi-step task, comprising:
entering a task request, by a task requestor, into a task schedule maintained by a scheduling system, the task request comprising information used by an event notification system to generate first and second task request messages having instructions used by an individual in the performance of the multi-step task;
sending, by the event notification system, the first task request message to the individual, the first task request message comprising a set of two or more options from which the individual can select in order to perform a first portion of the multi-step task;
the individual selecting an option from the set of options comprising the first task request message;
detecting, at the event notification system, that an option in the set of two or more options comprising the first task request message is selected, determining that the first portion of the multi-step task is complete and sending a task status message to the task requestor that includes information corresponding to the performance of the selected option;
sending, by the event notification system, the second task request message to the individual, the second task request message comprising a set of two or more options from which the individual can select in order to perform a second portion of the multi-step task, at least one of the options in the set of two or more options in the second task request message being different from any of the options in the set of two or more options comprising the first task request message;
the individual selecting an option from the set of two or more options comprising the second task request message; and
detecting, at the event notification system, that the option is selected from the set of two or options comprising the second task request message, determining that the second portion of the multi-step task is complete and sending a task status message to the task requestor that includes information corresponding to the performance of the selected option.

2. The method of claim 1, further comprising the event notification system determining that the multi-step task is complete and causing the multi-step task to be cancelled from the task schedule.

3. The method of claim 1, wherein the multi-step task comprises preparing a patient for transportation and transporting the patient from a current first location to a second location in accordance with instructions comprising any of the task request messages.

4. The method of claim 2, wherein the multi-step task further comprises the patient being transported from the second location to the first location or to another location in accordance with instructions comprising any of the task request messages.

5. The method of claim 1, wherein either the first or the second task request message comprises one or more of a patient identity, a patient location, a location identity to which the patient is transported, a time that the patient is scheduled to arrive at the identified location, patient preparation information, a transportation means, who is transporting the patient and whether the transportation is two-way or one-way.

6. The method of claim 1, wherein the set of two or more options comprising the first or second task request messages comprise any two or more of an indication that one or more porter are needed to transport a patient, an indication of the means by which the patient is to be transported, an indication that a nurse is transporting the patient, and an indication that the patient is not ready for transportation, an indication that the patient is in transit, and indication that the patient is delivered to the identified location, and that the patient is delayed.

7. The method of claim 1, wherein the information corresponding to the performance of the task comprises information indicating that a step in the multi-step task request is waiting to be performed, is in the process of being performed or is completed.

8. The method of claim 1, further comprising not detecting at the event notification system that an option comprising either the first or the second task request message is selected within a selected period of time, and the event notification system escalating the task request message.

9. The method of claim 8, wherein the task request message is escalated to another individual as determined by the event notification system.

10. The method of claim 9, further comprising the task request message to the individual is cancelled.

11. Method of coordinating the performance of a scheduled multi-step task between a first and a second individual, comprising:
entering a task request, by a task requestor, into a task schedule maintained by a scheduling system, the task request comprising information used by an event notification system to generate a first task request message having instructions used by the first individual to perform a first portion of the multi-step task and to generate a second task request message having instructions used by the second individual to perform a second portion of the multi-step task;
sending, by the event notification system, the first task request message to the first individual, the first task request message comprising a set of two or more options from which to select in order to perform the first portion of the multi-step task;
the first individual selecting an option from the set of two or more options comprising the first task request message;
detecting, at the event notification system, that an option in the set of two or more options comprising the first task request message is selected and sending a task status message to the task requestor that includes information corresponding to the performance of the selected option;
sending, by the event notification system, the second task request message to the second individual, the second task request message comprising a set options from which to select in order to perform the second portion of the multi-step task, at least one option in the set of options being different than any of the options included in the set of options comprising the first task request message;
the second individual selecting an option from the set of two or more options in the second task request message; and
detecting, at the event notification system, that an option in the set of options comprising the second task request message is selected and sending a task status message to the task requestor that includes information corresponding to the performance of the selected option.

12. The method of claim 11, further comprising the event notification system detecting that the second one of at least two options comprising the second set of at least two options is selected by the second individual, and sending a task status message to the requestor indicating that all steps in the task are completed.

13. The method of claim 12, further comprising the event notification system determining that all steps in the multi-step task are complete and sending a message to the scheduling system that has information which causes the scheduling system to delete the multi-step task from the task schedule it maintains.

14. The method of claim 11, wherein the multi-step task comprises preparing a patient for transportation and transporting the patient from a current first location to a second location in accordance with instructions comprising any of the task request messages.

15. The method of claim 11, wherein the multi-step task further comprises the patient being transported from the second location to the first location or to another location in accordance with instructions comprising any of the task request messages.

16. The method of claim 11, wherein either the first or the second task request message comprises one or more of a patient identity, a patient location, a location identity to which the patient is transported, a time that the patient is scheduled to arrive at the identified location, patient preparation information, a transportation means, who is transporting the patient and whether the transportation is two-way or one-way.

17. The method of claim 11, wherein the set of two or more options comprising the first or second task request messages comprise any two or more of an indication that one or more porter are needed to transport a patient, an indication of the means by which the patient is to be transported, an indication that a nurse is transporting the patient, and an indication that the patient is not ready for transportation, an indication that the patient is in transit, and indication that the patient is delivered to the identified location, and that the patient is delayed.

18. The method of claim 11, wherein the information corresponding to the performance of the task comprises information indicating that a step in the multi-step task request is waiting to be performed, is in the process of being performed or is completed.

19. The method of claim 11, further comprising not detecting at the event notification system that an option comprising either the first or the second task request message is selected within a selected period of time, and the event notification system escalating the task request message.

20. The method of claim 19, wherein the task request message is escalated to another individual as determined by the event notification system.

21. The method of claim 20, further comprising the task request message to the individual is cancelled.

22. A method for coordinating the performance of a multi-step task, comprising:
entering a task request, by a task requestor, into an event notification system, the task request comprising information used by the event notification system to generate first and second task request messages having instructions used by an individual in the performance of the multi-step task;
sending, by the event notification system, the first task request message to the individual, the first task request message comprising a set of two or more options from which the individual can select in order to perform a first portion of the multi-step task;
the individual selecting an option from the set of options comprising the first task request message;
detecting, at the event notification system, that an option in the set of two or more options comprising the first task request message is selected, determining that the first portion of the multi-step task is complete and sending a task status message to the task requestor that includes information corresponding to the performance of the selected option;
sending, by the event notification system, the second task request message to the individual, the second task request message comprising a set of two or more options from which the individual can select in order to perform a second portion of the multi-step task, at least one of the options in the set of two or more options in the second task request message being different from any of the options in the set of two or more options comprising the first task request message;
the individual selecting an option from the set of two or more options comprising the second task request message; and
detecting, at the event notification system, that the option is selected from the set of two or options comprising the second task request message, determining that the second portion of the multi-step task is complete and sending a task status message to the task requestor that includes information corresponding to the performance of the selected option.
